(19) United States
(12) Patent Application Publication
(10) Pub. No.: US 2023/0083730 A1
(43) Pub. Date: Mar. 16, 2023

(54) Composition with Anti-Aging Properties and the Selection Method thereof

(71) Applicant: Hemix Biotech Limited, Hongkong (CN)

(72) Inventors: Sheung Kin Ken Wong, Hong Kong (HK); Xuelai Wang, Hong Kong (HK); Leung Kris Ying, Hong Kong (HK)

(21) Appl. No.: 17/902,931

(22) Filed: Sep. 5, 2022

Related U.S. Application Data
(60) Provisional application No. 63/240,410, filed on Sep. 3, 2021.

(51) Int. Cl. A61K 8/41; A61K 8/34; A61Q 19/08

(52) U.S. Cl. CPC A61K 8/41; A61K 8/347; A61Q 19/08

(57) ABSTRACT

The present invention relates to the field of biomedical technology, and particularly relates to a composition with anti-aging properties, which comprises spermidine and resveratrol. The invention provides a method for selecting a composition with anti-aging properties, and the anti-aging properties of the composition were confirmed by lifespan analysis, cell viability analysis and staining analysis using materials such as Caenorhabditis elegans and mouse embryonic fibroblasts etc. The composition can be used to improve life-span, as anti-aging drugs and nutritional products. The invention also discloses the preferred concentrations of spermidine and resveratrol in the composition, where the preferred concentration of spermidine is 200 μM and the preferred concentration of resveratrol is 10 μM. The method as provided by the invention is versatile, and the provided compositions can be used in drug preparation and epidemic medicine.

### CROSS-REFERENCE TO RELATED APPLICATIONS

This application claims priority to U.S. Provisional Patent Application No. 63/240,410, entitled “Combined use of spermidine and resveratrol as anti-aging product”, filed on Sep. 3, 2021, the disclosure of which is incorporated by reference in its entirety.

### BACKGROUND OF THE INVENTION

The present invention is in the technical field of biopharmaceuticals. More particularly, the present invention relates to a composition with anti-aging properties and a selection method thereof, wherein the composition comprises spermidine and resveratrol.

Population aging and aging-related diseases are the major topics facing by the researchers worldwide. Researchers have spent a lot of effort to come up with anti-aging therapies and drugs. According to the prior art references, researchers have discovered that spermidine can promote cellular lifespan by inducing cellular autophagy. For example, an article of 2009 by Eisenberg, T., et al. (Eisenberg, T., et al., Induction of autophagy by spermidine promotes longevity. Nat Cell Biol, 2009. 11(11): p. 1305-14.) reported that administration of spermidine markedly extended the lifespan of yeast, flies and worms, and human immune cells. Another example is an article in 2010 by Morselli, E., et al. (Morselli, E., et al., Caloric restriction and resveratrol promote longevity through the Sirtuin-1-dependent Cell Death Dis, 2010. 1: p. e10.) showed that transgenic expression of Sirtuin-1 induces autophagy in human cells in vitro and in Caenorhabditis elegans in vivo. According to the conclusions of Morselli, E. et al., autophagy is universally required for the lifespan-prolonging effects of caloric restriction and pharmacological Sirtuin-1 activators.

According to the prior art literature, the effective concentrations of spermidine or resveratrol to extend the lifespan of Caenorhabditis elegans are 200 μM and 100 μM, respectively. As shown in the prior art literature, spermidine and resveratrol induce autophagy by distinct pathways. Another article of Morselli, E., et al. in 2011 (Morselli, E., et al. , E., et al., Spermidine and resveratrol induce autophagy by distinct pathways converging on the acetylproteome. J Cell Biol, 2011. 192(4): p. 615-29.) showed that induction of autophagy by resveratrol requires the nicotinamide adenine dinucleotide-dependent deacetylase sirtuin 1 (SIRT1). In the article of Morselli, E., et al. in 2011, it also shown that the acetylase inhibitor spermidine stimulates autophagy independent of SIRT1 in human and yeast cells as well as in nematodes.

Data from the prior art references show that caloric restriction has been shown to be able to improve the health of most organisms, including humans. However, adopting a lifestyle of caloric restriction or intermittent fasting is difficult for most people. Therefore, calorie restriction mimetics or chemicals which are able to induce autophagy and thus produce health-improving effects similar to those of calorie restriction have been studied and developed as drugs for the purpose of anti-aging.

As reported in the literature of the prior art, spermidine and resveratrol have been used as caloric restriction mimetics and have been tested in animal studies and clinical trials. For example, an article in 2006 by Baur, J. A., et al. (Baur, J. A., et al., Resveratrol improves health and survival of mice on a high-calorie diet. Nature, 2006. 444(7117): p. 337- 42.) showed that resveratrol (3,5,4′-trihydroxystilbene) extends the lifespan of diverse species including Saccharomyces cerevisiae, Caenorhabditis elegans and Drosophila melanogaster. In another article of 2016 by Eisenberg, T., et al. (Eisenberg, T., et al., Cardioprotection and lifespan extension by the natural polyamine spermidine. Nat Med, 2016. 22(12): p. 1428-1438.), it was noted that oral supplementation of the natural polyamine spermidine extends the lifespan of mice and exerts cardioprotective effects, reducing cardiac hypertrophy and preserving diastolic function in old mice.

The health-promoting effect of resveratrol depends on the stimulation of SIRT1 and AMPK, which leads to protein deacetylation and induction of autophagy. On the other hand, spermidine induces autophagy by inhibiting the activity of acetyltransferases, including the EP300 gene. In addition, spermidine can also inhibit mTORC1 and activate AMPK, thereby promoting the induction of autophagy.

Although data of prior art literatures show that spermidine and resveratrol have potential anti-aging effects and can induce cellular autophagy. However, the synthetic effects of the combined use of spermidine and resveratrol, especially on extending biological lifespan, have not been reported and disclosed in the prior art.

### SUMMARY OF THE INVENTION

According to one aspect the present invention, a method is provided for selecting composition having anti-aging properties, said method comprising the following steps:
Step 1: confirming the life-span enhancing effect of the composition using the Caenorhabditis elegans life-span analysis method;
Step 2: confirming the effect of the composition on up-regulating the expression level of an autophagy marker gene using an autophagy marker gene qPCR analysis;
Step 3: confirming the effect of the composition on restoring cell viability by analysis of mouse embryonic fibroblasts using cell viability analysis;
Step 4: confirming the effect of the composition on restoring cell viability by analyzing human bone marrow mesenchymal stem cells using cell viability assay; and
Step 5: confirming the effect of the composition on reducing senescent cells of by bone marrow-derived macrophage staining analysis.

According to another aspect of the present invention, the inventors provided a composition having anti-aging properties comprising spermidine and resveratrol. The composition comprising spermidine and resveratrol can significantly increase biological longevity relative to spermidine or resveratrol alone, and can be used as anti-aging drugs and nutritional products.

According to another aspect of the present invention, the inventors identified preferred concentrations of spermidine and resveratrol in the composition, wherein the preferred concentration of spermidine is 200 μM and the preferred concentration of resveratrol is 10 μM.

According to another aspect of the present invention, the inventors identified a preferred ratio of 20:1 of spermidine to resveratrol in the composition.

According to another aspect of the present invention, the inventors provided a method for preventing and treating diseases associated with aging with the composition comprising spermidine and resveratrol.

According to another aspect of the present invention, the inventors provided a method for using composition comprising spermidine and resveratrol for the preparations of anti-aging drugs.

### BRIEF DESCRIPTION OF DRAWINGS

The patent or application file contains at least one drawing executed in color. Copies of this patent or patent application publication with color drawing(s) will be provided by the Office upon request and payment of the necessary fee.

Embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regards, the description taken with the drawings makes apparent to those skilled in the how embodiments of the invention may be practiced.

In the drawings:

FIG. 1a is a graph shows the effects of resveratrol alone, spermidine alone, and the combined use of spermidine and resveratrol on the survival of Caenorhabditis elegans, where the four plots represent, from left to right, the survival curve of Caenorhabditis elegans treated with a control, 200 μM spermidine alone, 10 μM resveratrol alone, and a combined use of 200 μM spermidine and 10 μM resveratrol, respectively.

FIG. 1b is a table shows the n-values and the percentage increase in area under the curve for the four plots in FIG. 1a. con stands for control group, R stands for 10 μM resveratrol, S stands for 200 μM spermidine, and S+R stands for 10 μM resveratrol with 200 μM spermidine.

FIG. 2 is a bar chart shows the effect of expression of autophagy marker genes in wild-type mouse embryonic fibroblasts (MEFs) after treatment with resveratrol alone, spermidine alone, and with a combined use of spermidine and resveratrol. The autophagy marker genes were Atg7, Atg14, Nbr1, Ulk1, Ulk2 and Wdr45, and the results of qPCR analysis of these six autophagy marker genes are shown in the figure. The bars from left to right represent the fold difference after wild-type MEFs received two days of treatment using the control, 1 μM spermidine alone, 10 μM resveratrol alone, and combined use of 1 μM spermidine with 10μM resveratrol, respectively. * indicates a p-value less than or equal to 0.05, ** indicates a p-value less than or equal to 0.01, *** indicates a p-value less than or equal to 0.001, and **** indicates a p-value less than or equal to 0.0001.

FIG. 3 is a bar chart shows the cell viability of irradiated wild-type mouse embryonic fibroblasts (MEFs) treated with the combined use of spermidine and resveratrol. Specifically, the cell viability of wild-type MEFs irradiated by a 5 Gy dose receiving two days of treatment with the combined use and compared to a control group that did not receive treatment. * indicates a p-value less than or equal to 0.05 and ** indicates a p-value less than or equal to 0.01.

FIG. 4 is a bar chart shows the cell viability of wild-type human bone marrow mesenchymal stem cells (MSCs) after treatment with resveratrol alone, spermidine alone, and with the combined use of spermidine and resveratrol. The bars from left to right represent the cell viability of wild-type MSCs treated with control, spermidine alone, resveratrol alone and combined use of spermidine with resveratrol, respectively. * indicates that the p-value is less than or equal to 0.05.

FIG. 5a is the staining result showing the degree of senescence and the effect of M2-type macrophage polarization in irradiated mouse bone marrow-derived macrophages (BMDMs) treated with a combined use of spermidine and resveratrol. Specifically, they are the results of senescence-associated β-galactosidase staining analysis of mouse BMDMs irradiated with 10 Gy gamma dose after treatment with the combined use of spermidine and resveratrol. The top image in FIG. 5a is the original staining image, and the bottom image is the original image with the background removed by post-processing.

FIG. 5b is a bar chart shows the degree of senescence and M2-type macrophage type polarization effect of irradiated mouse bone marrow-derived macrophages (BMDMs) treated with the combined use of spermidine and resveratrol on. Specifically, they are the percentage of senescent cells in mouse BMDMs treated with the combined use of spermidine and resveratrol after gamma irradiation at a dose of 10 Gy.

FIG 5c is the staining result that shows the degree of senescence and the M2-type macrophage polarization in irradiated mouse bone marrow-derived macrophages (BMDMs) treated with the combined use of spermidine with resveratrol. Specifically, they are the results of immunofluorescence staining analysis of mouse BMDMs treated with the combined use of spermidine and resveratrol after gamma irradiation at a dose of 10 Gy. The blue channel indicates the results of DNA label DAPI staining and the far-red channel represents the results of CD206 staining for M2-type anti-inflammatory macrophage marker. The top image in FIG. 5c is the original image of the staining results, and the bottom image is the original image after post-processing to remove the background.

FIG. 6 is the flow chart shows steps of the method according to the present invention for selecting composition with anti-aging properties, comprising the six steps as follows, namely (1) confirming the effect of the composition on increasing biological lifespan by using the Caenorhabditis elegans lifespan analysis; (2) confirming the effect of composition on upregulating the expression level of autophagy marker genes by using the autophagy marker gene qPCR analysis; (3) confirming the effect of the composition on restoring cell viability by analyzing mouse embryonic fibroblasts using the cell viability analysis; (4) confirming the effect of the composition on restoring cell viability by analyzing human bone marrow mesenchymal stem cells using cell viability analysis; and (5) confirming the effect of the composition on reducing senescent cells by bone marrow-derived macrophage staining analysis.

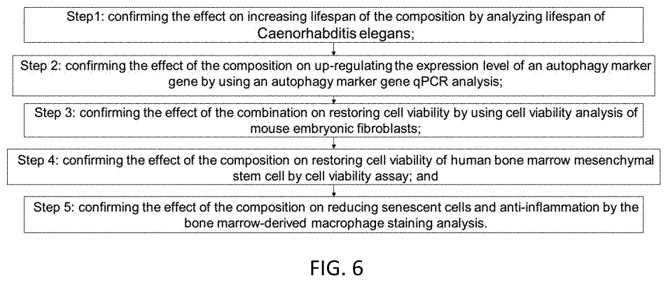

LIST OF ABBREVIATIONS

| | |
|---|---|
| SIRT1 | Sirtuin 1 protein |
| C. elegans | Caenorhabditis elegans |
| AMPK | AMP-activated protein kinase |
| EP300 | EP300 gene |
| mTORC1 | Mammalian Target of Rapamycin Complex 1 |
| MEFs | Mouse Embryonic Fibroblasts |
| MSCs | Mesenchymal Stem Cells |
| BMDMs | Bone Marrow-Derived Macrophage |
| M2 | M2 Polarization |
| CD206 | Mannose receptor (Cluster of Differentiation 206, |
| | CD206) |
| NGM | Nematode Growth Medium |
| OP50 | OP50 Strain |
| LB | Lysogeny Broth |
| LMNA | Lamin A/C |
| PBS | Phosphate Buffered Saline |
| Trypsin-EDTA | Trypsin-EDTA |
| EDTA | Ethylenediaminetetraacetic acid |
| DMEM | Dulbecco's Modified Eagle's Medium |
| HEPES | 4-(2-hydroxyethyl)-l-piperazineethanesulfonic acid |
| FBS | Fetal Bovine Serum |
| DEPC | Diethylpyrocarbonate |
| DNase | Deoxyribonuclease |
| qPCR | Real-time Polymerase Chain Reaction |
| mRNA | Messenger Ribonucleic Acid |
| cDNA | Complementary DNA |
| RNA | Ribonucleic Acid |
| DNA | Deoxyribonucleic Acid |
| ACK | (ACK) Lysing buffer |
| M-CSF | Macrophage Colony-Stimulating Factor |
| MTS | MTS(Owen's reagent) |
| PBSTr | 0.1% Triton X-100 in PBS |
| DAPI | 4′,6-diamidino-2-phenylindole, DAPI Staining |
| TRIzol | TRIzol ™ Reagent |
| Eppendorf | Eppendorf tube |
| rcf | Relative centrifugal field |
| Delta-Delta-Ct | 2-ΔΔCT method; 2-ΔΔCT value |
| p value | p-value (Statistical significance) |

### DETAILED DESCRIPTION OF THE INVENTION

The present invention provides a method that allows rapid selection of composition with anti-aging properties, by utilizing Caenorhabditis elegans, primary mouse embryonic fibroblasts (MEFs), human bone marrow mesenchymal stem cells (MSCs), and primary mouse bone marrow-derived macrophages, respectively. According to the method of selecting, the inventors provided a composition with anti-aging properties comprising spermidine and resveratrol with life-extending, anti-aging and anti-inflammatory effects. In the composition, the preferred concentration of spermidine is 10 μM and the preferred concentration of resveratrol is 200 μM.

In the process of carrying out the method of selection according to the present invention, the inventors confirmed that the composition comprising spermidine and resveratrol had a significantly better effect on life span extension in Caenorhabditis elegans than the treatment with spermidine or resveratrol alone. The composition upregulated the expression level of autophagy marker genes in embryonic fibroblasts (MEFs), increased cell viability, and protected cells from senescence due to DNA damage. The effect on enhancing cell viability of human bone marrow mesenchymal stem cells (MSCs) of the composition was significantly better than the treatment with spermidine or resveratrol alone. According to the prior art, stem cell exhaustion is one of the important hallmarks of aging; the composition protects cells from aging caused by DNA damage, and therefore prevents aging induced by stem cell exhaustion. The composition according to the present invention prevents senescence of macrophages when DNA is damaged, as well as upregulates the expression level of the M2-type anti-inflammatory macrophage marker CD206. According to the prior art, Macrophage is an important immune cell to regulate inflammation. Senescence of macrophage is linked to macrophage dysfunction and inflammation. The composition of the present invention can prevent macrophage senescence when DNA is damaged, as well as can upregulate the expression level of the M2-type anti-inflammatory macrophage marker CD206, and thus exhibits an anti-aging effect.

The present invention will be further described below in connection with embodiments. It should be understood that the embodiments are intended to illustrate the invention only, but are not intended to limit the scope of protection of the present invention. Where specific conditions are not indicated in the following embodiments, they are performed according to conventional conditions or with reference to the manufacturer's protocols. The instruments or reagents used, where the manufacturer is not specified, are conventional products available commercially.

### Step 1—Confirm the Life-Span Enhancing Effect of a Composition Comprising Spermidine and Resveratrol by Analyzing the Lifespan of Caenorhabditis elegans

According to an embodiment of the present invention, i.e., a method for selecting a composition having anti-aging properties, step 1 of the method is to confirm the life-span enhancing effect of the composition by using Caenorhabditis elegans lifespan analysis. For chemical treatment in Caenorhabditis elegans, four batches of chemical with different ingredients and content were added to the nematode growth medium (NGM) dishes. The NGM with agarose was autoclaved and then cooled down to below 65° C. The NGM was then poured into 60 mm dishes and incubated at room temperature overnight. The solidified NGM dishes were stored at 4° C. Escherichia coli OP50 strain was used as the food source for C. elegans. A single colony of OP50 was incubated in 200 mL of lysogeny broth (LB) medium at 37° C. overnight. The OP50 was then killed by incubation at 65° C. for 30 minutes. The dead OP50 was spread throughout the surface of the NGM dishes. The NGM dishes with dead OP50 were stored at 4° C. for up to 2 weeks. The synchronization of C. elegans was carried out by the bleaching method.

20 L4 larvae were picked to each 60 mm dish with different chemical treatment and cultured at 20° C. They were monitored every day, and the number of living, dead and missing C. elegans were recorded. The C. elegans were transferred to fresh Petri dishes every 2 days to separate the target worms from their offspring and to ensure that they had enough food source. At the end, the Caenorhabditis elegans lifespan analysis was carried out for the four groups of Caenorhabditis elegans treated with different ingredients and contents.

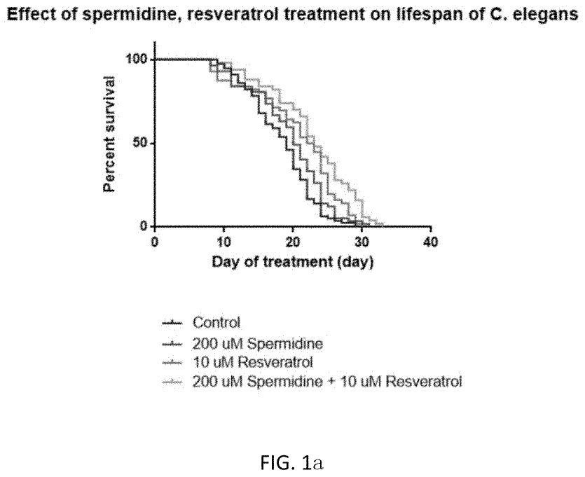

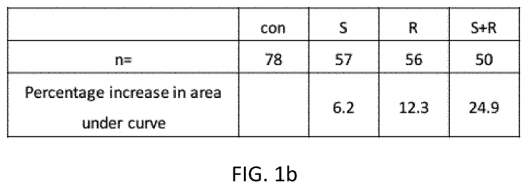

The ingredients and concentration of the four different batches of chemical are: a control blank, 200 μM spermidine alone, 10 μM resveratrol alone, and a combined use of 200 μM spermidine and 10 μM resveratrol. Referring to FIG. 1a, after 30-40 days of treatment and measurement, Caenorhabditis elegans treated with four different ingredients and levels of spermidine and resveratrol exhibited different life cycles. The four life-cycle plots in FIG. 1a correspond to four different ingredients and levels of spermidine and resveratrol, from left to right: control blank, 200 μM spermidine alone, 10 μM resveratrol alone, and combined use 200 μM spermidine and 10 μM resveratrol.

It is obvious from FIG. 1a, the life span of Caenorhabditis elegans treated with 200 μM spermidine alone and 10 μM resveratrol alone was higher than that of the control blank, and the life span of Caenorhabditis elegans treated with the composition comprising 200 μM spermidine and 10 μM resveratrol was higher than that of the two ingredients alone.

Referring to FIG. 1b, the table of FIG. 1b shows the n number and the percentage increase in the area under the survival curve of Caenorhabditis elegans. As seen in FIG. 1b, the percentage survival of Caenorhabditis elegans treated with the composition comprising 200 μM spermidine and 10 μM resveratrol was significantly higher than that of the two ingredients alone.

Upon the step 1 above, the inventors confirmed the beneficial effect of the composition comprising 200 μM spermidine and 10 μM resveratrol, which significantly increase the life span by the Caenorhabditis elegans life-span analysis.

### Step 2—Confirming the Cell Viability Enhancing Effect of the Composition Comprising Spermidine and Resveratrol by Autophagy Marker Gene qPCR Analysis

According to an embodiment of the present invention, i.e., a method for selecting a composition having anti-aging properties, step 2 of the invention is to confirm the cell viability enhancing effect of the composition comprising spermidine and resveratrol using autophagy marker gene qPCR analysis. The inventors utilized mouse embryonic fibroblasts (MEFs) as the subject of study for qPCR analysis of autophagy marker genes.

According to step 2 of embodiment of the present invention, mouse embryo fibroblasts (MEFs) were isolated for the study and analysis of next step. Heterozygous (LMNA^{G609G/+}) mice were set for mating. The date of pregnancy of mice was identified by checking for vaginal plug in the morning following the day of mating. If the vaginal plug was found, the mouse was considered to be pregnant for 0.5 days (E0.5). When the embryos reached E12.5 to E13.5, the pregnant mice were sacrificed, and the embryos were isolated from the mice inside tissue culture hood using sterile utensils. The embryos were placed in phosphate buffered saline (PBS). Their head and liver were removed. A portion of the head was used for genotyping to identify the genotype of each embryo. The body of each embryo was transferred to 1 mL of 0.1% Trypsin-EDTA solution in a well of 12-well plate. Using sterile scissors, embryos were cut into small pieces. The 12-well plate was then placed in a 37° C. incubator for 10 minutes. Followed by vigorous pipetting of the embryos in Trypsin-EDTA solution until the embryos wholly dissolved into the solution. The 12-well plate was then placed in 37° C. incubator for 5 minutes. The homogenized solution was then transferred to 9 mL of Gibco's High Glucose Dulbecco's Modified Eagle's Medium (DMEM) supplemented with sodium bicarbonate (3.7 g/L), HEPES (6 g/L), 10% fetal bovine serum (FBS) and penicillin-streptomycin (100 units/mL). The isolated MEFs were considered to be at passage 0 (P0). P3 MEFs were used in experiments as they have optimal cellular responses to stimuli. Primary mouse embryonic fibroblasts (MEFs) were cultured in Gibco's High Glucose Dulbecco's Modified Eagle's Medium (DMEM) supplemented with sodium bicarbonate (3.7 g/L), and 10% fetal bovine serum (FBS).

Primary mouse embryonic fibroblasts (MEFs) at passage 3 were seeded were selected for further analysis and experiments. In addition, at the end, the qPCR analysis was used to confirm that the composition containing spermidine and resveratrol had the effect of upregulating the mRNA expression levels of autophagy marker genes. Primary mouse embryonic fibroblasts (MEFs) at passage 3 were and cultured in 10-cm dish and treated with control, resveratrol, spermidine and combined treatment for 2 days. For chemical treatment of primary mouse embryonic fibroblasts, four batches of chemical with different ingredients and content were added to the nematode growth medium (NGM) dishes. The ingredients and concentration of the four different batches of chemical are: a control blank, 200 μM spermidine alone, 10 μM resveratrol alone, and a combined use of 200 μM spermidine and 10 μM resveratrol.

The cells were washed twice with ice-cold phosphate buffered saline (PBS), and 1 mL TRIzol was added. After incubation at room temperature for 5 minutes, the samples were collected into Eppendorf tubes. The supernatants were collected to Eppendorf tubes. 200 μl of chloroform was added to each sample, and the tubes were shaken vigorously by hand for 15 seconds. The samples were incubated at room temperature for 5 minutes and then centrifuged at 4° C. at 12000 rcf for 15 minutes. After centrifugation, the solution was separated into 3 layers. 450 μl of the clear top layer was collected, and 500 μl of isopropanol was added. After incubation at room temperature for 10 minutes, the samples were centrifugated at 4° C. at 12000 rcf for 10 minutes. RNA pellets would be visible at the bottom of the tubes. The RNA pellets were washed by 75% ethanol in diethylpyrocarbonate (DEPC) water.

For qPCR, the samples were centrifugated at 4° C. at 7500 rcf for 5 minutes. The ethanol was removed, and the samples were air-dried until the RNA pellets turned transparent. Then the RNA pellets were re-suspended with DEPC water. The concentration of RNA was measured by NanoDrop. 2 μg of RNA was used for DNase digestion and reverse transcription. For DNase digestion, 2 μg of RNA was used for each reaction using Promega RQ1 Rnase-Free Dnase following the manufacturer's protocol. DNase digestion removes the DNA contaminant in the RNA samples, which prevents the amplification of genomic DNA in qPCR. The DNase digested RNA samples were then used for reverse transcription using Thermo Scientific's High Capacity cDNA Reverse Transcription Kit with RNase Inhibitor following manufacturer's protocol. The transcribed cDNA was diluted 5 times with Milli-Qwater. Then the samples were ready for qPCR. For qPCR process, 0.5-1 μl of cDNA was used for each 10 μl reaction. The Delta-Delta-Ct value was calculated by the qPCR machine based on the signal of target genes normalized with housekeeping genes. Atg7, Atg14, Nbr1, Ulk1, Ulk2 and Wdr45 are important autophagy markers.

According to the embodiment of the present invention, qPCR analysis was carried out to analyze the expression level of these six autophagy marker genes after treatment with four different batches of chemical with different ingredients and concentration of spermidine and resveratrol. The ingredients and concentration of the four different batches of chemical are: a control blank, 1 μM spermidine alone, 10 μM resveratrol alone, and a combined use of 1 μM spermidine and 10 μM resveratrol, respectively.

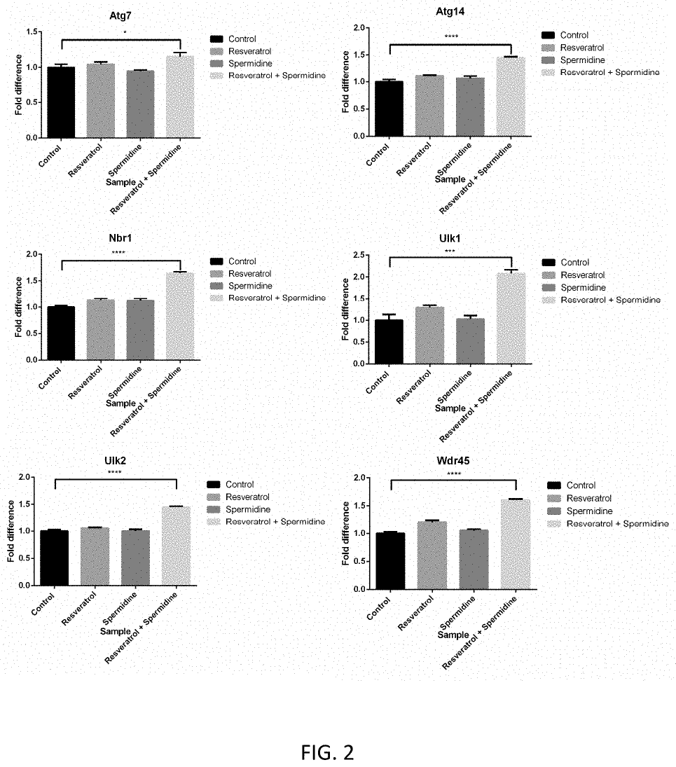

Referring to FIG. 2, the six bars in the bar chart correspond to the fold difference of the six autophagy marker genes, namely Atg7, Atg14, Nbr1, Ulk1, Ulk2, and Wdr45, respectively. The four bars in each bar chart represents, from left to right, a control blank, treatment with 1 μM spermidine alone, treatment with 10 μM resveratrol alone, and treatment with a combined use of 1 μM spermidine with 10 μM resveratrol. In FIG. 2, * indicates a p-value less than or equal to 0.05, ** indicates a p-value less than or equal to 0.01, *** indicates a p-value less than or equal to 0.001, and **** indicates a p-value less than or equal to 0.0001. As seen from FIG. 2, the results of qPCR analysis showed that for the six important autophagy marker genes, namely Atg7, Atg14, Nbr1, Ulk1, Ulk2, and Wdr45, their mRNA expression levels were upregulated to the highest extend in the case of treatment with the combined use of resveratrol and spermidine. Since the combined use of the spermidine and resveratrol significantly induced cellular autophagy, it was further confirmed that the composition comprising spermidine and resveratrol extended lifespan. Based on step 2 of the method of the present invention, the inventors confirmed that the composition comprising spermidine and resveratrol upregulated the expression levels of autophagy marker genes in embryonic fibroblasts (MEFs), increased cell viability, and protected cells from aging due to DNA damage.

### Step 3—Confirming the Effect of Restoring Cell Viability for the Composition by Analyzing Cell Viability of Primary Mouse MEFs after Irradiation

According to an embodiment of the present invention, i.e., a method for selecting a composition having anti-aging properties, step 3 of the invention is to confirm the cell viability enhancing effect of a composition comprising spermidine and resveratrol using a cell viability assay. The inventors confirmed the effect of restoring cell viability of the composition comprising spermidine and resveratrol by using the composition. In step 3 of the method according to the present invention, the inventors utilized primary mouse embryonic fibroblasts (MEFs) for the experiment and analysis.

Cells were cultured in 96-well plate and CellTiter 96® AQueous One Solution Cell Proliferation Assay (MTS) was used to carry out the cell viability assay. For primary mouse embryonic fibroblasts (MEFs), passage 3 MEFs were seeded and cultured in 96-well plate and treated with control or combined treatment of resveratrol with spermidine for 2 days after receiving 5 Gy gamma irradiation.

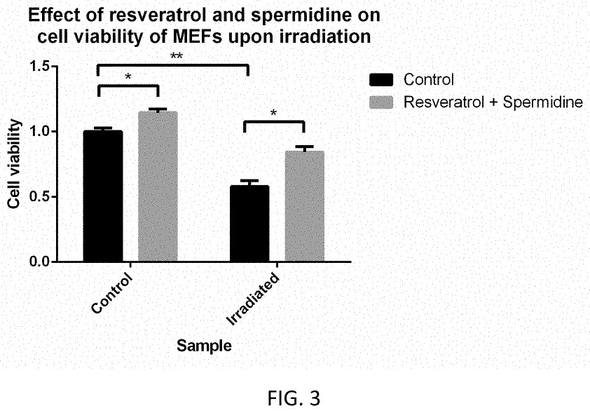

Referring to FIG. 3, the two columns on the left side show the difference in cell viability between unirradiated embryonic fibroblasts treated with and without the combined use of resveratrol and spermidine, and the two columns on the right side show the difference in cell viability between irradiated embryonic fibroblasts treated with and without the combined use of resveratrol and spermidine. The results show that treatment on both unirradiated and irradiated embryonic fibroblasts with the combined use of resveratrol and spermidine increased the cell viability of MEFs.

The results as presented in FIG. 3 show that gamma irradiation causes DNA damage in MEFs, and that DNA damage induces cellular senescence and cell viability of senescent cells decreases. By comparing the difference in cell viability of embryonic fibroblasts treated with and without the composition comprising resveratrol and spermidine, the step 3 of the method according to the present invention confirmed that the composition comprising resveratrol and spermidine can restore cell viability, and it is followed that the composition has the anti-aging effects.

### Step 4—Confirming the Anti-Aging Effect of a Composition by Human Bone Marrow Mesenchymal Stem Cell Viability Analysis

According to an embodiment of the present invention, i.e., a method for selecting a composition having anti-aging properties, step 4 of the method is to confirm the anti-aging effect of a composition comprising spermidine and resveratrol using human bone marrow mesenchymal stem cell viability assay. To further confirm that the composition comprising spermidine and resveratrol enhances cell viability, the inventors utilized human bone marrow mesenchymal stem cells (MSCs) for the further experiments and analysis.

Firstly, cells were cultured in 96-well plate and CellTiter 96® AQueous One Solution Cell Proliferation Assay (MTS) was used to carry out the cell viability assay. For human mesenchymal stem cells (MSCs), they were seeded and cultured in 96-well plate and treated with control, resveratrol, spermidine or combined treatment for 2 days.

Human bone marrow mesenchymal stem cells (MSCs) were treated with four batches of chemical having different ingredients and concentrations of spermidine and resveratrol for two days under identical experimental conditions. The ingredients and concentrations of spermidine and resveratrol of the four different chemicals are: control blank, spermidine alone, resveratrol alone, and a combined use of spermidine and resveratrol.

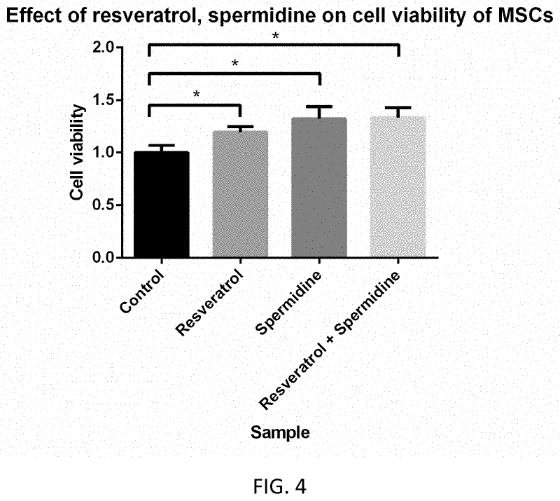

Referring to FIG. 4, the bar chart represents from left to right the cell viability of human bone marrow MSCs treated with a) control blank, b) resveratrol alone, c) spermidine alone, and d) a combined use of spermidine and resveratrol, respectively. * indicates a p value less than or equal to 0.05. The results show that either resveratrol alone or spermidine alone can enhance cell viability. However, treatment with the combined use of spermidine and resveratrol was superior to the enhancement brought by resveratrol alone or spermidine alone. Therefore, in the step 4 of the method according to the invention, the inventors confirmed that the composition comprising spermidine and resveratrol can enhance the cell viability of human bone marrow mesenchymal stem cells (MSCs), and the enhancement effect is significantly better than the treatment with spermidine alone or resveratrol alone.

### Step 5—Confirming the Anti-Aging Effect of the Composition by Staining Analysis

According to an embodiment of the present invention, i.e., a method for selecting a composition having anti-aging properties, step 5 of the method is to confirm the anti-aging effect of a composition comprising spermidine and resveratrol. In the step 5 of the method according to the present invention, by using bone marrow-derived macrophages, the inventors confirmed the effect of restoring cell viability of the composition comprising spermidine and resveratrol by using two different approaches, namely senescence-related β-galactosidase staining analysis and immunofluorescence staining analysis. According to the embodiment of the present invention, adult wildtype mouse was sacrificed and its body was soaked with 75% ethanol. Epiphyses of femur and tibia were then removed and bone marrow was flushed out by syringe with 2-3 mL PBS for each piece of bone. The bone marrow was centrifuged at 200×g for 5 minutes at 4° C. and supernatant was removed. 1 mL of ACK (Ammonium-Chloride-Potassium) lysing buffer was used to re-suspend cells. After incubating for 1 min to remove erythrocytes, 10 mL DMEM with 10% FBS, 1% P/S was added and the bone marrow was centrifuged 200×g for 5 minutes at 4° C. After centrifugation, the supernatant was removed and the cells were re-suspended in 1 mL DMEM with 10% FBS, 1% P/S. The cells were then filtered through 100 μm cell strainer and transferred to 10 cm dish. 9 mL DMEM with 10% FBS, 1% P/S was added to the 10 cm dish. 10 ng/mL M-CSF was also added to the medium. The cells were cultured at incubator at 37° C. and 5% CO2. The primary mouse bone marrow-derived macrophages (BMDMs) were used for experiments after 6 days of culture.

According to embodiment of the present invention, in order to confirm the anti-aging effect of a composition comprising, combined use of spermidine and resveratrol was used to treat bone marrow-derived macrophages irradiated with a 10 Gy dose of gamma irradiation, and as compared to a control blank after two days of treatment. Two different staining methods were used to confirm the anti-aging effect of the composition comprising spermidine and resveratrol.

The first staining analysis method as used is senescence-associated β-galactosidase staining analysis, where a senescence-associated β-galactosidase staining kit was used. Cells were fixed with paraformaldehyde for 10 minutes and then washed twice with PBS. The slides were then incubated with the staining solution at 37° C. overnight. The next day, the slides were washed twice with PBS, and then mounted with 40% glycerol in PBS. The stained cells were observed under a light microscope, and it could be observed that senescent cells were stained blue, while proliferating cells were not stained. The percentage of senescent cells was quantified by counting the number of stained and unstained cells. Gamma irradiation was used to induce damage to macrophage DNA, which triggers cellular senescence.

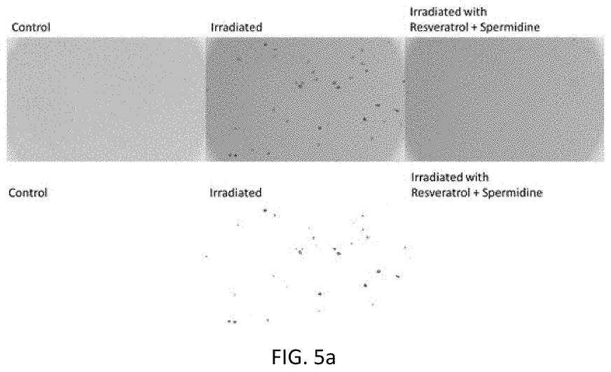

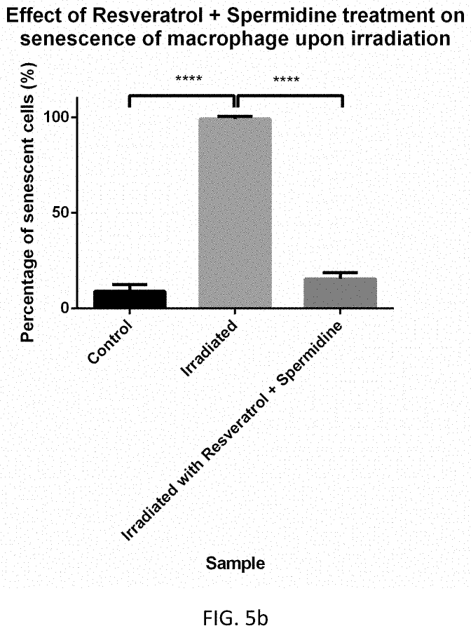

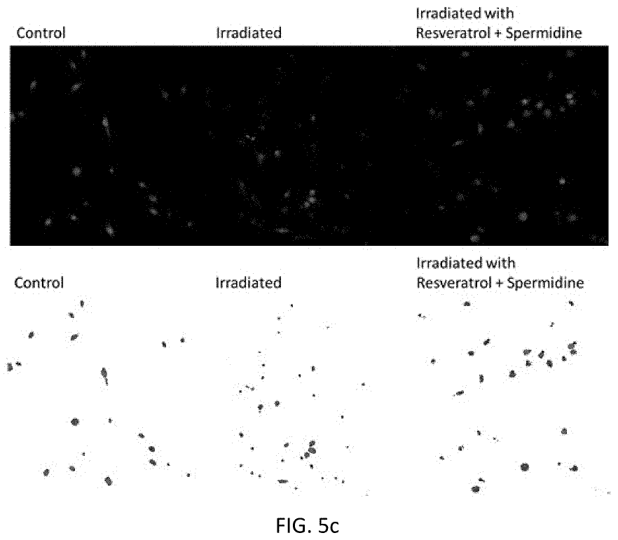

Referring to the FIGS. 5a and 5b, wherein FIG. 5a shows the staining analysis results of senescence-associated β-galactosidase staining for the irradiated mouse bone marrow-derived macrophages, after treatment with a composition comprising resveratrol and spermidine, wherein the image on the left side is the staining results of the control blank, the one in the middle is the staining results of the irradiated group, and the one on the right side is the staining results of treatment with a composition comprising resveratrol and spermidine after irradiation. The bar chart of FIG. 5b shows the percentage of senescent cells in irradiated mouse bone marrow-derived macrophages, after treatment with the composition comprising resveratrol and spermidine composition, wherein the bar from left to right represents the percentage for the control blank, the irradiated group, and the group treated with the composition comprising resveratrol and spermidine after irradiation.

It is obvious from the staining effect of FIG. 5a that gamma irradiation leads to DNA damage in macrophages, and that DNA damage increases the amount of cellular senescence. According to the staining results of FIG. 5a, it was confirmed that the composition comprising resveratrol and spermidine, as provided by the present invention, has a beneficial effect in reducing senescent cells. Thus, the anti-aging properties of the composition comprising spermidine and resveratrol were confirmed.

The second staining analysis method as used is the immunofluorescence staining analysis. The BMDMs were cultured in 8-well chamber slides and treated with control or resveratrol spermidine combined treatment for 2 days after receiving 10 Gy gamma irradiation. The slides were washed twice with PBS and incubated in PBSTr (0.1% Triton X-100 in PBS) for 10 minutes. Then the cells were blocked with 5% fetal bovine serum (FBS) in PBSTr for 1 hour at room temperature. Primary antibodies diluted in 5% FBS in PBSTr were added and incubated at 4° C. overnight. On the following day, the cells were washed three times using PBSTr, incubation for 10 minutes each time. Secondary antibodies diluted in 5% FBS in PBSTr were added and incubated at room temperature for 1 hour. The cells were again washed three times using PBSTr, followed by PBS for two times. The slides were mounted with SlowFade Gold antifade reagent. Then the slides were ready to be analyzed under a confocal microscope.

Referring to FIG. 5c, the staining results as shown are the immunofluorescence staining analysis of irradiated mouse bone marrow-derived macrophages, after treatment with a composition comprising resveratrol and spermidine, where the staining results for the control group are shown on the left, the staining results for the irradiated group are shown in the middle, and the staining results for treatment with a composition comprising resveratrol and spermidine are shown on the right. The blue channel represents DNA label DAPI staining and the far-red channel represents M2 type anti-inflammatory macrophage marker CD206 staining.

As seen from the staining effect of FIG. 5c, gamma irradiation leads to a decrease in the expression level of macrophage M2-type anti-inflammatory macrophage marker CD206, but after treatment with the composition comprising resveratrol and spermidine, the expression of macrophage M2-type anti-inflammatory macrophage marker CD206 increases significantly. The results of FIG. 5c demonstrate that the composition comprising resveratrol and spermidine according to the present invention can upregulate the expression of the macrophage M2 type anti-inflammatory macrophage marker CD206, and thus can be used to treat inflammation inducible epidemics, and can also be used to prepare drugs for the treatment of inflammation inducible epidemics. Upon the step 5 of the method according to the present invention, the inventors confirmed that the composition comprising spermidine and resveratrol was effective in reducing senescent cells and in up-regulating the expression of M2 anti-inflammatory macrophage marker CD206 of the macrophage. Thus both the anti-aging and anti-inflammatory properties of the composition comprising spermidine and resveratrol were confirmed.

Although the invention has been shown and described with respect to a certain embodiment, it is apparent that this invention can be embodied in many different forms and that many other modifications and variations are possible without departing from the spirit and scope of this invention.

Moreover, while exemplary embodiments have been described herein, one of ordinary skill in the art will readily appreciate that the exemplary embodiments set forth above are merely illustrative in nature and should not be construed as to limit the claims in any manner. Rather, the scope of the invention is defined only by the appended claims and their equivalents, and not, by the preceding description.

### REFERENCES

1. Eisenberg, T., et al., Induction of autophagy by spermidine promotes longevity. Nat Cell Biol, 2009. 11(11): p. 1305-14.

2. Wood, J. G., et al., Sirtuin activators mimic caloric restriction and delay ageing in metazoans. Nature, 2004. 430(7000): p. 686-9.

3. Morselli, E., et al., Caloric restriction and resveratrol promote longevity through the Sirtuin-1-dependent induction of autophagy. Cell Death Dis, 2010. 1: p. e10.

4. Morselli, E., et al., Spermidine and resveratrol induce autophagy by distinct pathways converging on the acetylproteome. J Cell Biol, 2011. 192(4): p. 615-29.

5. Baur, J. A., et al., Resveratrol improves health and survival of mice on a high-calorie diet. Nature, 2006. 444(7117): p. 337-42.

6. Eisenberg, T., et al., Cardioprotection and lifespan extension by the natural polyamine spermidine. Nat Med, 2016. 22(12): p. 1428-1438.

7. Schwarz, C., et al., Safety and tolerability of spermidine supplementation in mice and older adults with subjective cognitive decline. Aging (Albany, N.Y.), 2018. 10(1): p. 19-33.

8. Berman, A. Y., et al., The therapeutic potential of resveratrol: a review of clinical trials. NPJ Precis Oncol, 2017. 1.

9. Lagouge, M., et al., Resveratrol improves mitochondrial function and protects against metabolic disease by activating SIRT1 and PGC-1alpha. Cell, 2006. 127(6): p. 1109-22.

10. Price, N. L., et al., SIRT1 is required for AMPK activation and the beneficial effects of resveratrol on mitochondrial function. Cell Metab, 2012. 15(5): p. 675-90.

11. Madeo, F., et al., Caloric Restriction Mimetics against Age-Associated Disease: Targets, Mechanisms, and Therapeutic Potential. Cell Metab, 2019. 29(3): p. 592-610.

12. Pietrocola, F., et al., Spermidine induces autophagy by inhibiting the acetyltransferase EP300. Cell Death Differ, 2015. 22(3): p. 509-16.

13. Marino, G., et al., Regulation of autophagy by cytosolic acetyl-coenzyme A. Mol Cell, 2014. 53(5): p. 710-25.

The invention claimed is:

1. A method for selecting a composition having anti-aging properties, wherein the method comprising the steps of:
(1) confirming the effect on increasing lifespan of the composition by analyzing lifespan of Caenorhabditis elegans;
(2) confirming the effect of the composition on up-regulating the expression level of an autophagy marker gene by using an autophagy marker gene qPCR analysis;
(3) confirming the effect of the composition on restoring cell viability by using cell viability analysis of mouse embryonic fibroblasts;
(4) confirming the effect of the composition on restoring cell viability of human bone marrow mesenchymal stem cell by cell viability assay; and
(5) confirming the effect of the composition on reducing senescent cells and anti-inflammation by the bone marrow-derived macrophage staining analysis.

2. The method for selecting a composition having anti-aging properties according to claim 1, wherein the composition of anti-aging properties comprising spermidine and resveratrol.

3. The method for selecting a composition having anti-aging properties according to claim 2, wherein the optimal concentration of spermidine is 200 μM and the optimal concentration of resveratrol is 10 μM.

4. The method for selecting a composition having anti-aging properties according to claim 2, wherein the optimal ratio of spermidine to resveratrol is 20:1.

5. The method for selecting a composition having anti-aging properties according to claim 2, wherein the optimal concentration of spermidine is 1 μM and the optimal concentration of resveratrol is 10 μM.

6. The method for selecting a composition having anti-aging properties according to claim 2, wherein the optimal ratio of spermidine to resveratrol is 1:10.

7. A composition having anti-aging properties, wherein the composition comprising spermidine and resveratrol.

8. The composition having anti-aging properties according to claim 7, wherein the optimal concentration of spermidine is 200 μM and the optimal concentration of resveratrol is 10 μM.

9. The composition having anti-aging properties according to claim 7, wherein the optimal concentration of spermidine is 1 μM and the optimal concentration of resveratrol is 10 μM.

10. The composition having anti-aging properties according to claim 7, wherein the optimal ratio of spermidine to resveratrol is 20:1 to 1:10.
